(19) United States
(12) Patent Application Publication
(10) Pub. No.: US 2017/0254023 A1
(43) Pub. Date: Sep. 7, 2017

(54) DISPERSIBLE WIPE

(71) Applicants: Georgia-Pacific Consumer Products LP, Atlanta, GA (US); Lenzing Aktiengesellschaft, Lenzing (AT)

(72) Inventors: Daniel W. Sumnicht, Hobart, WI (US); Stephanie P. Boyce, De Pere, WI (US)

(73) Assignees: Georgia-Pacific Consumer Products LP, Atlanta, GA (US); Lenzing Aktiengesellschaft, Lenzing (AT)

(21) Appl. No.: 15/449,167

(22) Filed: Mar. 3, 2017

Related U.S. Application Data
(60) Provisional application No. 62/303,651, filed on Mar. 4, 2016.

(51) Int. Cl. D21H 27/00; D21H 21/20; D21H 17/37; A61Q 19/00; A61K 8/02; A61K 8/73; A61K 8/81; D21H 27/30; D21H 13/08

(52) U.S. Cl. CPC D21H 27/005; D21H 27/30; D21H 21/20; D21H 17/375; D21H 13/08; A61K 8/0208; A61K 8/731; A61K 8/8158; A61Q 19/007; A61K 2800/20

(57) ABSTRACT

A water dispersible wipe includes a web including at least 10 weight % (wt. %) of a fibrillated regenerated cellulose microfiber (CMF) based on the total weight of the web, the remaining fibers of the web including natural cellulosic fibers; a wet strength agent disposed within the web; and a pH-modifying composition having a wetting pH disposed within the web; wherein the water dispersible wipe includes at least two webs and disperses upon exposure to an aqueous environment having a pH greater than the wetting pH and has a cross-direction (CD) wet tensile of at least 75 g/in after 24 hours as measured according to IVDA Standard Test WSP 110.4 (05).

### CROSS-REFERENCE TO RELATED APPLICATION

This application claims benefit of U.S. Provisional Patent Application Ser. No. 62/303,651, filed Mar. 4, 2016, the entire contents of which is incorporated herein in its entirety.

### TECHNICAL FIELD

The instant invention generally relates to wipes. More specifically, the instant invention relates to water dispersible wipes.

### BACKGROUND

Disposable wipe and tissue products are convenient, relatively inexpensive, sanitary, and easy to use. Personal care wipes are convenient because they are portable, suitable for travel, and versatile. Examples of disposable wipes include wet wipes (or wipers), e.g., baby wipes and cosmetic wipes. In addition to personal care wipes, disposable household wipes include kitchen-cleaning wipes and dusting wipes.

Although convenient, discarding of disposable wipes can be problematic when the wipe substrates are not biodegradable or not “flushable.” “Flushable” refers to an ability to evacuate a toilet. Wipe substrates that are not biodegradable or flushable can accumulate in landfills. However, even “flushable” wipe substrates may not be made of materials that are substantially water dispersible. In particular, a wipe substrate's ability to evacuate a toilet can be merely due to small size. Thus, wipes that do not disintegrate or substantially disperse in water have disadvantages because they can plug screens and jam pumps in sewage treatment plants.

One method for making dispersible wipes includes using regenerated cellulose fibers, or Lyocell fibers. PCT International Publication Number WO 95/35399 describes the manufacture of Lyocell fibers with an increased fibrillation tendency. PCT International Publication Number WO 2013/067557 describes the manufacture of dispersible wipes including Lyocell fibers, without an increased fibrillation tendency, and pulp.

Another method for making dispersible wet wipes includes hydroentangling fibrous webs including long fibers. Water jets impart energy to entangle long fibers. Product design should balance the desire for strength with the ability to disperse. More water jet energy increases the web strength, but may decrease dispersibility. Wet wipes made from hydroentangling may not be fully dispersible. Accordingly, there is a need for a highly dispersible, biodegradable wet wipe. It is to solving this problem the present invention is directed.

### SUMMARY OF THE INVENTION

The present invention is directed to water dispersible wipes. In one or more aspects of the invention, a water dispersible wipe includes a web including at least 10 weight % (wt. %) of a fibrillated regenerated cellulose microfiber (CMF) based on the total weight of the web, the remaining fibers of the web including natural cellulosic fibers; a wet strength agent disposed within the web; and a pH-modifying composition having a wetting pH disposed within the web; wherein the water dispersible wet wipe includes at least two webs and disperses upon exposure to an aqueous environment having a pH greater than the wetting pH and has a cross-direction (CD) wet tensile of at least 75 g/in after 24 hours as measured according to IVDA Standard Test WSP 110.4 (05). In another aspect, the CD wet tensile is at least 100 Win after 24 hours.

In other aspects, a water dispersible wet wipe includes a web including a CMF in a range from about 10 wt. % to about 100 wt. % based on the total weight of the web, the remaining fibers of the web including natural cellulosic fibers, and the CMF having a freeness of less than 175 milliliters (mL); a glyoxalated wet strength agent disposed within the web; and a pH-modifying composition having a wetting pH disposed within web; wherein the water dispersible wet wipe disperses upon exposure to an aqueous environment having a pH greater than the wetting pH and has a CD wet tensile of at least 200 grams/3 inch (g/3 in) as measured according to TAPPI Method T 576 pm-07. In another aspect, the CD wet tensile is at least 500 g/3 in.

Yet, in other aspects, a method of making a water dispersible wet wipe includes forming a web including CMF, a wet strength agent, and natural cellulosic fibers, the CMF being present in an amount of at least 10 wt. % based on the total weight of the web, and the remaining fibers of the web including natural cellulosic fibers; and disposing a pH-modifying composition having a wetting pH within the web; wherein the water dispersible wet wipe disperses upon exposure to an aqueous environment having a pH greater than the wetting pH and has a CD wet tensile of at least 200 grams/3 inch (g/3 in) as measured according to TAPPI Method T 576 pm-07. In another aspect, the CD wet tensile is at least 500 g/3 in.

Still yet, in other aspects, a water dispersible wipe includes a web including CMF in a range from about 10 wt. % to about 100 wt. % based on the total weight of the web, the remaining fibers of the web including natural cellulosic fibers, and the CMF having a freeness of less than 175 milliliters (mL); a glyoxalated wet strength agent disposed within the web; and a pH-modifying composition having a wetting pH disposed within the web; wherein the water dispersible wipe disperses upon exposure to an aqueous environment having a pH greater than the wetting pH and has a MD dry tensile to CD dry tensile ratio of about 1:1 to about 3:1.

It is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods, and systems for carrying out the present invention. It is important, therefore, that the claims be regarded as including such equivalent constructions insofar as they do not depart from the spirit and scope of the present invention.

Other advantages and capabilities of the invention will become apparent from the following description taken in conjunction with the examples showing aspects of the present invention.

### BRIEF DESCRIPTION OF THE DRAWINGS

The invention will be better understood and the above object as well as other objects other than those set forth above will become apparent when consideration is given to the following detailed description thereof. Such description makes reference to the annexed drawing wherein:

FIG. 1A is a light microscopy image (50×) of CMF formed in accordance with the present invention.

FIG. 1B is a light microscopy image (200×) of CMF formed in accordance with the present invention.

FIG. 1C is a light microscopy image (50×) of CMF produced from non-rapid fibrillating regenerated cellulose.

FIG. 1D is a light microscopy image (200×) of CMF produced from non-rapidly fibrillating regenerated cellulose.

FIG. 2A is a scanning electron micrograph (SEM) image of a handsheet made with 50% refined softwood fibers and 50% CMF.

FIG. 2B is a SEM image of a handsheet made with 100% CMF.

FIG. 3 is a graph illustrating wet tensile of handsheets wetted for 24 hours as a function of % CMF.

FIG. 4 is a graph comparing wet tensile of handsheets.

FIG. 5 is a graph showing dispersibility of handsheets.

FIG. 6 is a graph illustrating wet tensile decay of the wetted handsheets over time.

FIG. 7 is a graph showing MD wet tensile as a function of dry tensile in basesheets.

FIG. 8 is a graph illustrating the “shelf stability” of wet wipes.

### DETAILED DESCRIPTION OF THE INVENTION

For a fuller understanding of the nature and desired objects of this invention, reference should be made to the above and following detailed description taken in connection with the accompanying figures. When reference is made to the figures, like reference numerals designate corresponding parts throughout the several figures.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

As used herein, the articles “a” and “an” preceding an element or component are intended to be nonrestrictive regarding the number of instances (i.e. occurrences) of the element or component. Therefore, “a” or “an” should be read to include one or at least one, and the singular word form of the element or component also includes the plural unless the number is obviously meant to be singular.

As used herein, the terms “invention” or “present invention” are non-limiting terms and not intended to refer to any single aspect of the particular invention but encompass all possible aspects as described in the specification and the claims.

As used herein, the term “about” modifying the quantity of an ingredient, component, or reactant of the invention employed refers to variation in the numerical quantity that can occur, for example, through typical measuring and liquid handling procedures used for making concentrates or solutions in the real world. Furthermore, variation can occur from inadvertent error in measuring procedures, differences in the manufacture, source, or purity of the ingredients employed to make the compositions or carry out the methods, and the like. Whether or not modified by the term “about,” the claims include equivalents to the quantities. In one aspect, the term “about” means within 10% of the reported numerical value, or within 5% of the reported numerical value.

As used herein, the terms “percent by weight,” “% by weight,” and “wt. %” mean the weight of a pure substance divided by the total dry weight of a compound or composition, multiplied by 100. Typically, “weight” is measured in grams (g).

As used herein, the term “web” means a wet-laid, non-hydroentangled web.

As used herein, the term “Canadian Standard Freeness,” “freeness,” or “CSF” is a measure of the CMF surface area after fibrillation. Freeness is determined in accordance with Technical Association of the Paper and Pulp Industry (TAPPI) Standard T 227 OM-94 (Canadian Standard Method). Any suitable method of preparing the regenerated cellulose microfiber for freeness testing may be employed as long as the fiber or microfiber is well dispersed. For example, if the fiber is pulped at 5% consistency for a few minutes or more, i.e., 5-20 minutes before testing, the fiber is well dispersed for testing. Likewise, partially dried fibrillated regenerated cellulose microfiber can be treated for 5 minutes in a British disintegrator at 1.2% consistency to ensure proper dispersion of the fibers. All preparation and testing is done at room temperature and either distilled or deionized water is used throughout.

As used herein, the term “wet strength agent” means a chemical compound or composition that increases the wet tensile properties of a web.

As used herein, the term “water dispersible” means the ability of a wipe to readily break apart or disperse in water. A water dispersible wipe readily breaks apart due to the physical forces encountered during flushing in a common toilet, conveyance in a common wastewater system, and processing in a common treatment system.

As used herein, the term “CMF” is an abbreviation for “cellulose microfiber” or “CMF” that is obtained by mechanically agitating (or fibrillating) regenerated cellulose fibers. Agitating the fibers causes smaller “micro” fibers to shear off of the parent fiber. In some aspects, the agitated (or fibrillated) regenerated cellulose fibers are, e.g., Lyocell fibers (commercially available from Lenzing AG, Lenzing, Austria, under the brand TENCEL®).

Regenerated cellulose fibers, such as Lyocell, are made using the lyocell process. In the lyocell process, cellulose pulp is dissolved in a solvent with tertiary amine N-oxides (NMMO). The dope comprising water, cellulose, and NMMO is extruded through spinnerets into a coagulation bath to form regenerated cellulose fibers with a natural tendency to fibrillate. Technology is employed to minimize the fibrillation tendency so that fibers are useful for textiles. An example of the textile grade referenced herein is “Lyocell dull” grade, which has a small amount of mineral filler added to deluster the fiber appearance. In the wetted state, Lyocell dull grade is agitated to form CMF. Lyocell dull grade can be converted into CMF through extensive mechanical energy despite being designed for textiles.

Lyocell with enhanced tendency to fibrillate (“fast-fibrillating Lyocell”) is another CMF grade. Consequently, CMF prepared from fast-fibrillating Lyocell is of better quality and obtained with less than 50% of the energy input used for the textile grade (Lyocell dull grade). Fast-fibrillating Lyocell is used to make CMF used in webs, handsheets, and wipers described herein. The drop in freeness and the increase in scattering coefficient after mechanical agitation are used to assess fibrillation.

In one aspect of the present invention, a water dispersible wipe includes a web having at least 10 weight % (wt. %) of a fibrillated regenerated cellulose microfiber (CMF) based on the total weight of the web, the remaining fibers of the web include natural cellulosic fibers; a wet strength agent disposed within the web; and a pH-modifying composition having a wetting pH disposed within the web; wherein the water dispersible wipe disperses upon exposure to an aqueous environment having a pH greater than the wetting pH and has a cross-direction (CD) wet tensile of at least 75 g/in after 24 hours as measured according to INDA Standard Test WSP 110.4 (05). In another aspect, the CD wet tensile is at least 100 g/in after 24 hours.

The water dispersible wipes described herein can be wet wipes or dry wipes. The water dispersible wipes can include, for example, one ply (web), two plies (webs), three plies (webs), or more than three plies (webs). When the dry wipe is wetted, the wipe has a CD wet tensile of at least 75 g/in after 24 hours as measured according to INDA Standard Test WSP 110.4 (05). In another aspect, the CD wet tensile is at least 100 g/in after 24 hours.

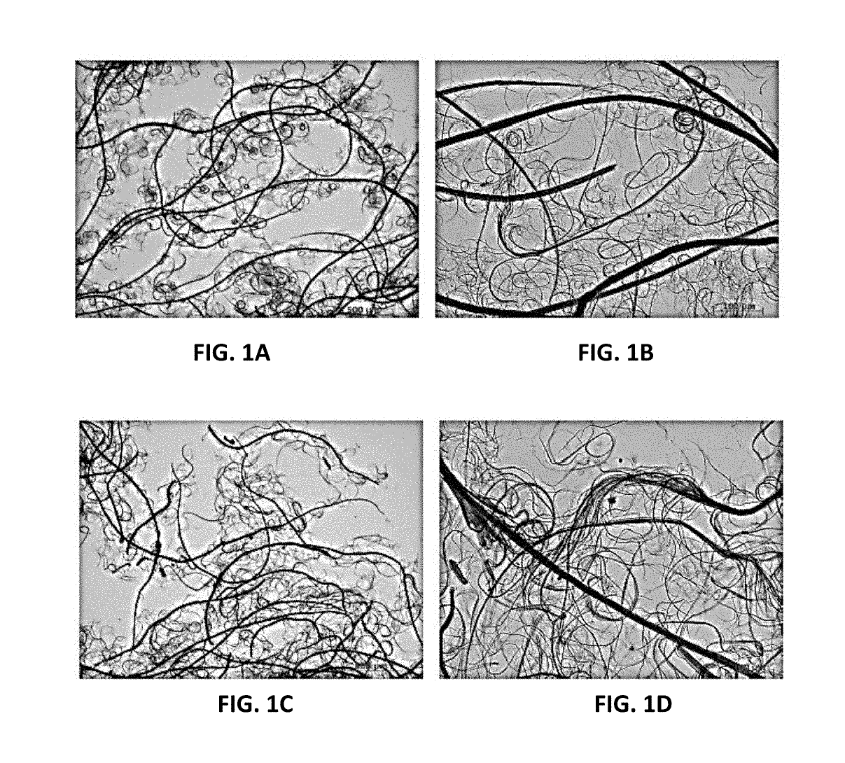

FIG. 1A and FIG. 1B are light microscopy images of CMF formed in accordance with the present invention at 50× and 200× magnification, respectively. For comparison, FIG. 1C and FIG. 1D are light microscopy images of CMF formed from non-rapid fibrillating regenerated cellulose. As shown, the rapidly fibrillating CMF (FIGS. 1A and 1B) have longer fiber length and curl than the non-rapidly fibrillating CMF (FIGS. 1C and 1D).

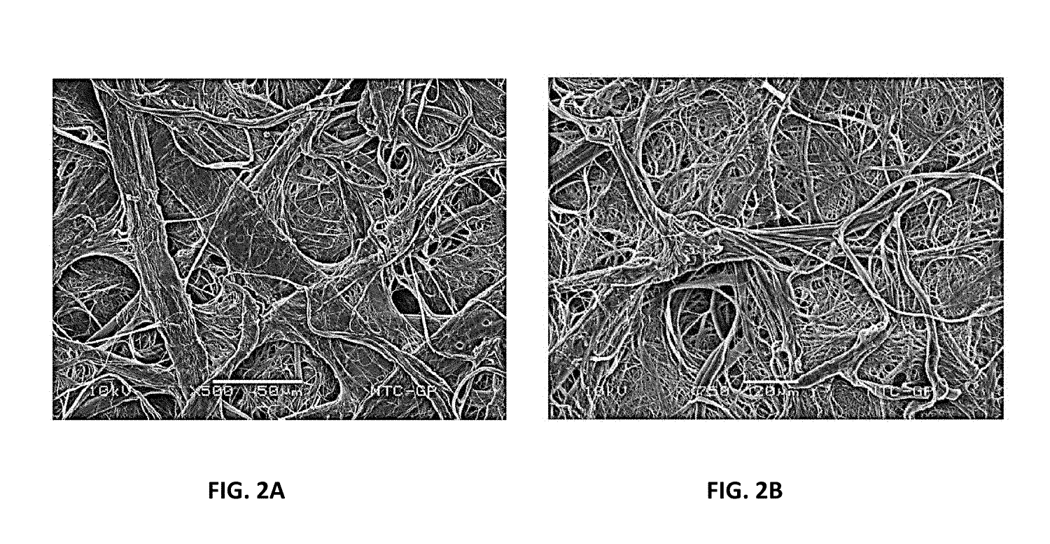

FIG. 2A is a SEM image of a handsheet made with 50% refined softwood fibers and 50% CMF, which illustrates the difference in size of the CMF and long, flat softwood fibers. Handsheets are prepared by “hand,” and, therefore, fiber is distributed uniformly in a round sheet. CMF that has sheared off of the parent regenerated cellulose fiber form an interconnected web around the softwood fibers. FIG. 2B is a SEM image of a handsheet made with 100% CMF, which illustrates a higher fiber population than FIG. 2A.

After fibrillation, the CMF retains a length-weighted average length of at least 2.0 mm. In one aspect, the length-weighted average length of the CMF after fibrillation is in a range from about 1.5 to about 5 mm. In another aspect, the length-weighted average length of the CMF after fibrillation is in a range from about 3 to about 4 mm. Yet, in another aspect, the length-weighted average length of the CMF is about or in any range from about 1.5, 2.0, 2.5, 3.0, 3.5, 4.0, 4.5, 5.0, 5.5, 6.0, 6.5, 7.0, 7.5, 8.0, 8.5, 9.0, 9.5, and 10.0 mm.

The average fiber length may be measured using a fiber-measuring instrument, for example, a kajaaniFiberLab™ instrument (commercially available from Metso Corporation, Helsinki, Finland) or a Fiber Quality Analyzer (commercially available from OpTest Equipment Inc., Hawkesbury, Ontario, Canada). For fiber length measurements, a dilute suspension of the fibers is prepared in a sample beaker. The instrument is operated according to the procedures recommended by the manufacturer. The report range for fiber lengths is set at the instrument's minimum value, for example, 0.07 mm, and a maximum value, for example, 7.2 mm. Fibers having lengths outside of the selected range are excluded. Three calculated average fiber lengths may be reported. The arithmetic average length is the sum of the product of the number of fibers measured and the length of the fiber divided by the sum of the number of fibers measured. The length-weighted average fiber length is the sum of the product of the number of fibers measured and the length of each fiber squared divided by the sum of the product of the number of fibers measured and the length the fiber. The weight-weighted average fiber length is the sum of the product of the number of fibers measured and the length of the fiber cubed divided by the sum of the product of the number of fibers and the length of the fiber squared. For microfibers, other optical techniques may be used.

The average diameter of the CMF is in a range from about 1 micrometer (micron) to about 20 microns. In one aspect, the average diameter of the CMF is in a range from about 5 microns to about 15 microns. In another aspect, the average diameter of the CMF is about or in any range from about 1, 2, 3, 4, 5, 6, 7, 8, 9, 10, 11, 12, 13, 14, 15, 16, 17, 18, 19, and 20 microns.

The CMF has a high surface area that creates greater friction compared to cellulose fibers, for example, Kraft wood fibers, alone. The surface area of the CMF is characterized by its “freeness” or “CSF” value. The CMF has a freeness (or CSF value) of less than 175 milliliters (mL). In one aspect, the CMF has a freeness in a range from about 10 mL to about 175 mL. In another aspect, the CMF has a freeness in a range from about 20 mL to about 60 mL. Yet, in another aspect, the CMF has a freeness about or in any range from about 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, and 170 mL.

Entanglement friction contributes to the wet tensile of a wet, fibrous web, which is described in, for example, Tejado, A. and Van de Ven, T., Materials Today, September 2010, Vol. 3, No. 9, which is incorporated herein in its entirety by reference. Entanglement friction, however, does not refer to entanglement caused by water jets used in hydroentangling. Rather, it refers to force exerted from one fiber to others at fiber crossings. CMF contributes to this friction due to a high population of low-coarseness fibrils that create a large number of fiber-fiber or fiber-fibril crossings. The strength contribution is proportional to the length of the fibrils and fibers, and is inversely proportional to the coarseness (weight per length) of the CMF fibrils and fibers. As Lyocell is fibrillated, more and more low-coarseness fibrils are generated, resulting in increasing fibrous surface area. Light scattering coefficient of sheets made with cellulose fibers and CMF provide a way to quantify surface area available for internal wet friction.

Scattering coefficient of sheets made with CMF, wood, or other fibers is proportional to unbonded surface area. The “scattering coefficient (S)” is determined in accordance with TAPPI test method T-425 om-01, the disclosure of which is incorporated herein in its entirety by reference. This method functions at an effective wavelength of 572 nm. The scattering coefficient (m²/kg) is the normalized value of scattering power to account for basis weight of the web. The “characteristic scattering coefficient” refers to the scattering coefficient of a standard web made from 100% of that fiber, excluding components that substantially alter the scattering characteristics of neat pulp such as fillers, and the like.

The CMF described herein demonstrates a characteristic scattering coefficient of less than 300 m²/kg. In another aspect, the CMF demonstrates a characteristic scattering coefficient in a range from about 100 to about 200 m²/kg. Yet in another aspect, the CMF demonstrates a characteristic scattering coefficient about or in any range from about 50, 60, 70, 80, 90, 100, 110, 120, 130, 140, 150, 160, 170, 180, 190, 200, 210, 220, 220, 230, 240, 250, 260, 270, 280, 290, and 300 m²/kg.

In addition to CMF, the web includes other natural cellulosic fibers, for example, pulp-derived papermaking fibers. Non-limiting examples of suitable cellulose fibers include any fiber incorporating cellulose as a major constituent. Suitable papermaking fibers include, but are not limited to, non-wood fibers or bast fibers, such as cotton fibers or cotton derivative fibers, abaca fibers, kenaf fibers, sabai grass fibers, flax fibers, esparto grass fibers, straw fibers, jute hemp fibers, bagasse fibers, milkweed floss fibers, ramie fibers, arundo donax fibers, and pineapple leaf fibers. Other suitable wood fibers, include, but are not limited to, fibers obtained from deciduous and coniferous trees, including softwood fibers, such as northern and southern softwood Kraft fibers; hardwood fibers, such as eucalyptus fibers, maple fibers, birch fibers, aspen fibers, and the like.

The natural cellulosic fibers can be present in the web in an amount in a range from about 10 wt. % to about 90 wt. % based on the total weight of the web. In another aspect, the natural cellulosic fibers are present in the web in an amount in a range from about 50 to about 80 wt. % based on the total weight of the web. Yet, in another aspect, the natural cellulosic fibers are present in the web in an amount about or in any range from about 10, 15, 20, 25, 30, 35, 40, 45, 50, 55, 60, 65, 70, 75, 80, 85, and 90 wt. % based on the total weight of the web.

The webs formed from the CMF and natural cellulosic fibers are substantially water dispersible and biodegradable. The webs can be prepared according to conventional processes known to those skilled in the art, including conventional wet pressing, through-air-drying (TAD), eTAD, Yankee/air-drying, ATMOS, NTT, UCTAD, and variations thereof. The webs also may be formed using a perforated belt to form a structured web, as described below.

For example, wet pressed webs can be made on a papermaking machine by first preparing and mixing the CMF and natural cellulosic fibers, wet strength agent, and any desired additional additives in a vat to produce a fiber slurry. The wet strength agent is dispersed throughout the web.

The wet strength agent can be any temporary wet strength agent capable of increasing the wet tensile of the web. One example of a suitable wet strength agent is a glyoxalated strength agent or a cationic glyoxalated strength agent, for example, cationic glyoxalated polyacrylamide (GPAM). Other aldehyde-containing wet strength agents can be used. Non-limiting examples of commercially available temporary wet strength agents include Bubond® 818 (Buckman Laboratories International, Inc., Memphis, Tenn.), HercoBond®1194 (Solenis LLC, Wilmington, Del.), and Fennorez® 98 or Fennorez® 110 (Kemira Chemicals, Inc.).

Other non-limiting examples of suitable wet strength agents include glyoxal; glutaraldehyde; uncharged chemical moieties, such as dialdehydes, aldehyde-containing polyols, uncharged aldehyde-containing polymers, cyclic ureas and mixtures thereof, aldehyde-containing cationic starch, or any combination thereof.

When present, the wet strength agent is added to the fibers and is present in the web in an amount in a range from about 0.01 to about 8 pounds per ton (lb/T). In some aspects, the wet strength agent is not added to the fibers. In one aspect, the wet strength agent is added to the fibers and present in the web in an amount of less than 6 lb/T. In another aspect, the wet strength agent is added to the fibers and present in the web in an amount about or in any range from about 0.01, 1, 2, 3, 4, 5, 6, 7, and 8 lb/T.

When the wet strength agent is an aldehyde-containing agent, e.g., a glyoxalated strength agent such as glyoxalated polyacrylamide, the molecular weight of the polymer is chosen to balance the wet tensile achieved and the extent of wet tensile decay. Lower molecular weight promotes greater decay while higher molecular weight may give higher wet tensile with less decay. The web may be prepared on a papermaking machine to produce a tissue basesheet. Because the tissue basesheet is prepared on a paper machine, it has fiber orientation (machine direction (MD) and cross direction (CD)). More than one tissue basesheet (ply), for example, two or more, can be combined to form a multi-ply wipe. One method of making the web involves diluting the CMF and natural cellulosic fibers to the desired consistency, adding the wet strength agent, and transferring through a centrifugal pump to a headbox. From the headbox, the fibrous mixture is deposited onto a moving foraminous wire, such as fourdrinier wire, to form a nascent web. Water can drain through the wire by use of vacuum and/or drainage elements. The nascent web can be compactively dewatered on a papermaking felt and dried on a Yankee dryer or compactively dewatered and applied to a rotating cylinder and fabric creped therefrom. Drying techniques include any conventional drying techniques, such as through drying, impingement air drying, Yankee drying and so forth.

Any suitable creping fabric with any desired pattern may be used to form a patterned web. The web also may be embossed to provide a desired pattern.

Another method of making the web includes using a perforated belt to produce a structured web or sheet. The use of a perforated polymeric belt makes it possible to obtain consolidated regions in the web, providing improved physical properties (e.g., bulk and caliper). A perforated belt bears a perforated pattern suitable for practicing the process of the present invention. In addition to perforations, the belt may have features, such as raised portions and/or recesses between perforations, if so desired. The perforations can be tapered to facilitate transfer of the web. The creping belt can include decorative features, such as geometric designs, floral designs, and so forth, formed by rearrangement, deletion, and/or a combination of perforations having varying sizes and shapes. A perforated polymer creping belt as described in U.S. Patent Application Publication No. 2010/0186913, entitled “Belt-Creped, Variable Local Basis Weight Absorbent Sheet Prepared With Perforated Polymeric Belt,” the disclosure of which is incorporated herein in its entirety by reference, can be used to produce a structured web.

Other methods/technologies that may be used to make the webs for the wipes described herein include eTAD methods, ATMOS methods, NTT methods, UCTAD methods, and variations thereof. Non-limiting examples of suitable methods are disclosed in the following U.S. patent applications and patents, which are incorporated herein in their entireties by reference: U.S. patent application Ser. No. 11/804,246 (Publication No. US 2008-0029235), entitled “Fabric Creped Absorbent Sheet with Variable Local Basis Weight” filed May 16, 2007, (Attorney Docket No. 20179; GP-06-11), now U.S. Pat. No. 7,494,563, which was based upon U.S. Provisional Patent Application Ser. No. 60/808,863, filed May 26, 2006; U.S. patent application Ser. No. 10/679,862 (Publication No. US-2004-0238135), entitled “Fabric Crepe Process for Making Absorbent Sheet”, filed Oct. 6, 2003 (Attorney Docket No. 12389; GP-02-12), now U.S. Pat. No. 7,399,378; U.S. patent application Ser. No. 11/108,375 (Publication No. US 2005-0217814), entitled “Fabric Crepe/Draw Process for Producing Absorbent Sheet”, filed Apr. 18, 2005 (Attorney Docket No. 12389P1; GP-02-12-1), which application is a continuation-in-part of U.S. patent application Ser. No. 10/679,862 (Publication No. US-2004-0238135), entitled “Fabric Crepe Process for Making Absorbent Sheet”, filed Oct. 6, 2003 (Attorney Docket No. 12389; GP-02-12), now U.S. Pat. No. 7,399,378; U.S. patent application Ser. No. 11/108,458 (Publication No. US 2005-0241787), entitled “Fabric Crepe and In Fabric Drying Process for Producing Absorbent Sheet”, filed Apr. 18, 2005 (Attorney Docket No. 12611P1; GP-03-33-1), now U.S. Pat. No. 7,442,278, which application was based upon U.S. Provisional Patent Application No. 60/563,519, filed Apr. 19, 2004; U.S. patent application Ser. No. 11/151,761 (Publication No. US 2005-0279471), entitled “High Solids Fabric Crepe Process for Producing Absorbent Sheet With In-Fabric Drying”, filed Jun. 14, 2005 (Attorney Docket No. 12633; GP-03-35), now U.S. Pat. No. 7,503,998 which was based upon U.S. Provisional Patent Application Ser. No. 60/580,847, filed Jun. 18, 2004; U.S. patent application Ser. No. 11/402,609 (Publication No. US 2006-0237154), entitled “Multi-Ply Paper Towel With Absorbent Core”, filed Apr. 12, 2006 (Attorney Docket No. 12601; GP-04-11), which application was based upon U.S. Provisional Patent Application No. 60/673,492, filed Apr. 21, 2005; U.S. patent application Ser. No. 11/104,014 (Publication No. US 2005-0241786), entitled “Wet-Pressed Tissue and Towel Products with Elevated CD Stretch and Low Tensile Ratios Made With a High Solids Fabric Crepe Process”, filed Apr. 12, 2005 (Attorney Docket No. 12636; GP-04-5), now U.S. Pat. No. 7,588,660, which application was based upon U.S. Provisional Patent Application No. 60/562,025, filed Apr. 14, 2004; and U.S. patent application Ser. No. 11/451,111 (Publication No. US 2006-0289134), entitled “Method of Making Fabric-Creped Sheet for Dispensers”, filed Jun. 12, 2006 (Attorney Docket No. 20079; GP-05-10), now U.S. Pat. No. 7,585,389, which application was based upon U.S. Provisional Patent Application No. 60/693,699, filed Jun. 24, 2005; U.S. patent application Ser. No. 11/678,669 (Publication No. US 2007-0204966), entitled “Method of Controlling Adhesive Build-Up on a Yankee Dryer”, filed Feb. 26, 2007 (Attorney Docket No. 20140; GP-06-1; U.S. patent application Ser. No. 11/901,599 (Publication No. US 2008-0047675), entitled “Process for Producing Absorbent Sheet”, filed Sep. 18, 2007 (Attorney Docket No. 12611P1D1; GP-03-33-D1), which application is a division of U.S. Pat. No. 7,442,278, U.S. patent application Ser. No. 11/901,673 (Publication No. US 2008-0008860), entitled “Absorbent Sheet”, filed Sep. 18, 2007 (Attorney Docket No. 12611P1D2; GP-03-33-D2), which application is a division of U.S. Pat. No. 7,442,278; U.S. patent application Ser. No. 12/156,820, (Publication No. US 2008-0236772), entitled “Fabric Crepe Process for Making Absorbent Sheet”, filed Jun. 5, 2008 (Attorney Docket No. 12389D2; GP-02-12B), now U.S. Pat. No. 7,588,661, which application is a division of U.S. Pat. No. 7,399,378; U.S. patent application Ser. No. 12/156,834, (Publication No. US 2008-0245492), entitled “Fabric Crepe Process for Making Absorbent Sheet”, filed Jun. 5, 2008 (Attorney Docket No. 12389D1; GP-02-12A), which application is a division of U.S. Pat. No. 7,399,378; and U.S. patent application Ser. No. 12/286,435, (Publication No. US 2009-0038768), entitled “Process for Producing Absorbent Sheet”, filed Sep. 30, 2008 (Attorney Docket No. 12611P1D3; GP-03-33-D3), which application is a division of U.S. Pat. No. 7,442,278.

The webs can be wet laid and formed without hydroentangling. Although hydroentangling processes can provide strong wet wipes, these specific processes require substantial investment and may reduce the dispersibility of wipers. Therefore, webs are formed without hydroentangling and with CMF that are both water dispersible and biodegradable. The webs formed using the CMF and natural cellulosic fibers described provide the desired wet tensile strength because of their inherent physical properties. Therefore, hydroentangling processes to impart strength are not needed.

The webs formed have a basis weight in a range from about 20 to about 60 grams per square meter (gsm) before plying. A single ply, for example, has a basis weight in a range from about 20 to about 60 gsm. In one aspect, a single ply of the web has a basis weight in a range from about 25 to about 35 gsm. In another aspect, each ply of the web has a basis weight about or in any range from about 20, 25, 30, 35, 40, 50, and 60 gsm.

Two or more plies of the webs can be combined to form a multi-ply wipe. The plies may be combined by any suitable methods known in the art. A multi-ply wipe can include, for example, two plies, three plies, or any number of the webs described herein.

The web is impregnated with a pH-modifying composition having a wetting pH to form the wet wipe. The water dispersible wipe disperses upon exposure to an aqueous environment having a pH greater than the wetting pH.

The wet strength agents maintain wet tensile of the web when the pH of the wetting composition is below the dispersing (wetting) pH. In one aspect, the wetting pH is in a range from about 3.5 to about 6.5. In another aspect, the wetting pH is in a range from about 4.5 to about 5.5. Yet, in another aspect, the wetting pH is about or in any range from about 3.5, 4.0, 4.5, 5.0, 5.5., 6.0, and 6.5.

The pH-modifying composition is added to the web in any amount desired sufficient to provide the desired pH. In some aspects, the pH-modifying composition is added to the web in a wetting composition. In other aspects, the pH-modifying composition is a wetting composition and the water dispersible wipe is a wet wipe. In one aspect, the wetting composition is present in the wet wipe in an amount in a range from about 65% to about 80% of the total weight of the wet wipe. In another aspect, the wetting composition is present in the wet wipe in an amount in a range from about 70% to about 75% of the total weight of the wet wipe. Yet in another aspect, the wetting composition is present in the wet wipe in an amount about or in any range from about 65, 70, 75, and 80% of the total weight of the wet wipe.

The pH-modifying composition includes a compound, for example, an acid, that reduces that pH to the wetting pH. Non-limiting examples of suitable acids include malic acid, citric acid, hydrochloric acid, sulfuric acid, or any combination thereof.

The pH-modifying composition can be a wetting composition including one or more additives. The wetting composition can be any solution, including, but not limited to, an aqueous solution comprising at least one additive. Non-limiting examples of suitable additives include a skin-care additive, an odor control agent, a disinfectant, a particulate, an antimicrobial agent, a preservative, a wetting agent, a cleaning agent, an emollient, a surface feel modifier, a fragrance, a fragrance solubilizer, an opacifier, a fluorescent whitening agent, an ultraviolet light absorber, or any combination thereof.

The wetting composition can be disposed onto or impregnated within the web by any method. Examples of such methods include, but are not limited to, soaking the web in the wetting composition and spraying the wetting composition onto the web.

The wet tensile of the water dispersible wipe is improved compared to a like wipe without the CMF and wet strength agent as described. Two tests are used to measure wet tensile, which depend on whether the wipe is a single ply made on a paper machine or plied into multiple plies to form a wet or dry wiper (e.g., two plies, three plies, or four plies).

When the single-ply web produced on the paper machine is tested, a three-inch wide sample is cut and tested in the cross direction (CD) or machine direction (MD) according to TAPPI Method T 576 pm-07, which is described briefly below. The dry paper three-inch strip of paper is moistened with standard water and tested immediately. The sample is folded into a loop, clamped in a special fixture termed a Finch Cup (Finch Cup Test), and then immersed in water. The Finch Cup, which is available from the Thwing-Albert Instrument Company (Philadelphia, Pa.), is mounted onto a tensile tester equipped with a 2.0-pound load cell with the flange of the Finch Cup clamped by the tester's lower jaw and the ends of tissue loop clamped into the upper jaw of the tensile tester. The sample is immersed in water that has been adjusted to a pH of 7.0±0.1 and the tensile is tested after a 5-second immersion time at a constant pull rate of 2 inches per minute. Values are divided by two, as appropriate, to account for the loop. The standard water contains sufficient hardness to simulate typical municipal water systems. The Finch Cup Test provides an indication of the decrease in tensile strength after wetting, and can be used to evaluate the single-ply web directly after production on the paper machine. Since the web is saturated with water, as opposed to the pH-modifying composition, this test can provide an indication of the interim wet strength of the unfinished product. Standard water solution, Part number R3489000-10F, is available from Fisher Scientific Company.

In one aspect, the CD wet tensile of the water dispersible wipe before plying immediately after being wetted is at least 200 g/3 in. In another aspect, the CD wet tensile of the water dispersible wipe before plying immediately after being wetted is at least 500 g/3 in. In one aspect, the CD wet tensile of the water dispersible wipe before plying immediately after being wetted is in a range from about 800 to about 1300 g/3 in. In another aspect, the CD wet tensile of the water dispersible wipe before plying immediately after wetting is about or in any range from about 200, 250, 300, 350, 400, 450, 500, 550, 600, 650, 700, 750, 800, 850, 900, 950, 1000, 1050, 1100, 1150, 1200, 1250, and 1300 g/3 in.

A web's geometric mean tensile (Tensile GM or GMT) is a measure of the tensile that takes into account the MD tensile strength and the CD tensile strength (both dry). The GMT is calculated as the square root of the product of the MD and CD tensile strengths. The dry CD and MD tensile strengths are measured according to TAPPI method T 576 pm-07.

According to one or more aspects, the CD dry tensile of a single web (basesheet) is less than the MD dry tensile. The ratio of MD tensile to CD tensile can be increased to facilitate dispensing from packaging and increase dispersibility. A lower CD tensile means that the sheet will have a lower overall strength, and therefore, have increased water dispersibility. In some aspects, the ratio of MD tensile to CD tensile is in a range from about 1:1 to about 3:1. In other aspects, the ratio of MD tensile to CD tensile is about 1.5:1 to about 3:1, about 2:1 to 3:1, or about 2.5:1 to 3:1.

In other aspects, a water dispersible wipe includes a web including CMF in a range from about 10 wt. % to about 100 wt. % based on the total weight of the web, with the remaining fibers of the web comprising natural cellulosic fibers, and the CMF having a freeness of less than 175 milliliters (mL). The web includes a glyoxalated wet strength agent disposed within the web and a pH-modifying composition having a wetting pH. The water dispersible wipe disperses upon exposure to an aqueous environment having a pH greater than the wetting pH and has a MD dry tensile to CD dry tensile ratio of about 1:1 to about 3:1.

When a single web is plied into a two- or three-ply wiper, or when testing a handsheet, the CD wet tensile is determined according to IVDA Standard Test WSP 110.4 (05). A one-inch strip cut from the sample in the cross-direction (CD) is tested after disposing a pH-modifying composition in the multi-ply wiper. The one-inch wide, pre-moistened sample is placed in jaws 4 inches apart and elongated at 12 inches per minute. This test is generally considered indicative of the tensile strength of a finished product. The web is disposed with a pH-modifying composition at a liquid add-on rate consistent with a wipe product, as opposed to being saturated with water.

Although the initial wet tensile of the multi-ply wet wipe decays within the first 24 hours, wet tensile then levels off and persists. In one aspect, the CD wet tensile of the multi-ply wipe is at least 75 Win after 24 hours. In another aspect, the CD wet tensile of the multi-ply wipe is at least 100 g/in after 24 hours. In another aspect, the CD wet tensile of the multi-ply wipe is in a range from about 75 to about 500 g/in after 24 hours. In one aspect, the CD wet tensile of the multi-ply wipe is in a range from about 100 to about 500 g/in after 24 hours. Yet, in another aspect, the CD wet tensile of the multi-ply wipe is about or in any range from about 75, 100, 150, 200, 250, 300, 350, 400, 450, and 500 g/in after 24 hours. Still yet, in another aspect, the CD wet tensile of the multi-ply wipe decays within the first 24 hours and then does not substantially decrease over the expected shelf life of the product.

The tensile energy absorption (TEA) refers to the work done when a specimen is stressed to rupture in tension under prescribed conditions as measured by the integral of the tensile strength over the range of tensile strain from zero to maximum strain. The TEA is expressed as energy per unit area (test span×width) of test specimen. The TEA may be measured according to TAPPI test method T 576 pm-07.

As indicated above, a variety of additives can be added to the wetting composition. Suitable additives include, but are not limited to: skin-care additives; odor control agents; disinfectants; particulates; antimicrobial agents; preservatives; wetting agents and cleaning agents such as detergents, surfactants, and some silicones; emollients; surface feel modifiers for improved tactile sensation (e.g., lubricity) on the skin; fragrance; fragrance solubilizers; opacifiers; fluorescent whitening agents; UV absorbers; pharmaceuticals; and pH control agents, such as malic acid or potassium hydroxide.

Skin-care additives provide one or more benefits to the user, such as a reduction in the probability of having diaper rash and/or other skin damage caused by fecal enzymes. These enzymes, particularly trypsin, chymotrypsin and elastase, are proteolytic enzymes produced in the gastrointestinal tract to digest food. In infants, for example, the feces tend to be watery and contain, among other materials, bacteria, and some amounts of undegraded digestive enzymes. These enzymes, if they remain in contact with the skin for any appreciable period of time, may cause an irritation that is uncomfortable in itself and can predispose the skin to infection by microorganisms. As a countermeasure, skin-care additives include, but are not limited to, the enzyme inhibitors and sequestrants.

A variety of skin-care additives can be added to the wetting composition and the pre-moistened wipes of the present invention or included therein. For example, skin-care additives in the form of particles can be added to serve as fecal enzyme inhibitors, offering potential benefits in the reduction of diaper rash and skin damage caused by fecal enzymes. Such materials can be used in the present invention, including reaction products of a long chain organic quaternary ammonium compound with one or more of the following clays: montmorillonite, bentonite, beidellite, hectorite, saponite, and stevensite.

Other known enzyme inhibitors and sequestrants can be used as skin-care additives in the wetting composition of the present invention, including those that inhibit trypsin and other digestive or fecal enzymes, and inhibitors for urease. For example, enzyme inhibitors and anti-microbial agents may be used to prevent the formation of odors in body fluids. Such inhibitors that can be incorporated into the wetting composition and the pre-moistened wipes of the present invention include transition metal ions and their soluble salts, such as silver, copper, zinc, ferric, and aluminum salts. The anion may also provide urease inhibition, such as borate, phytate, and the like. Compounds of potential value include, but are not limited to, silver chlorate, silver nitrate, mercury acetate, mercury chloride, mercury nitrate, copper metaborate, copper bromate, copper bromide, copper chloride, copper dichromate, copper nitrate, copper salicylate, copper sulfate, zinc acetate, zinc borate, zinc phytate, zinc bromate, zinc bromide, zinc chlorate, zinc chloride, zinc sulfate, cadmium acetate, cadmium borate, cadmium bromide, cadmium chlorate, cadmium chloride, cadmium formate, cadmium iodate, cadmium iodide, cadmium permanganate, cadmium nitrate, cadmium sulfate, and gold chloride. Other salts known to have urease inhibition properties include ferric and aluminum salts, such as the nitrates, and bismuth salts. Other urease inhibitors include hydroxamic acid and its derivatives; thiourea; hydroxylamine; salts of phytic acid; extracts of plants of various species, including various tannins, e.g., carob tannin, and their derivatives such as chlorogenic acid derivatives; naturally occurring acids such as ascorbic acid, citric acid, and their salts; phenyl phosphoro diamidate/diamino phosphoric acid phenyl ester; metal aryl phosphoramidate complexes, including substituted phosphorodiamidate compounds; phosporamidates without substitution on the nitrogen; boric acid and/or its salts, including especially, borax, and/or organic boron acid compounds; sodium, copper, manganese, and/or zinc dithiocarbamate; quinones; phenols; thiurams; substituted rhodanine acetic acids; alkylated benzoquinones; formarnidine disulphide; 1:3-diketones maleic anhydride; succinamide; phthalic anhydride; pehenic acid; N,N-dihalo-2-imidazolidinones; N-halo-2-oxazolidinones; thio- and/or acyl-phosphoryltnamide and/or substituted derivatives thereof, thiopyridine-N-oxides, thiopyridines, and thiopyrimidines; oxidized sulfur derivatives of diarninophosphinyl compounds; cyclotriphosphazatriene derivatives; bromo-nitro compounds; S-aryl and/or alkyl diamidophosphorothiolates; diarninophosphinyl derivatives; mono- and/or polyphosphorodiamide; alkoxy-1, 2-benzothaizin compounds; ortho-diaminophosphinyl derivatives of oximes; 5-substituted-benzoxathiol-2-ones; N(diammophosphinyl)arylcarboxamides; and the like.

Many other skin-care additives may be incorporated into the wetting composition, pre-moistened wet wipes, and dry webs during or post-manufacture, including, but not limited to, sun blocking agents and UV absorbers, acne treatments, pharmaceuticals, baking soda (including encapsulated forms thereof), vitamins and their derivatives such as Vitamins A or E, botanicals such as witch hazel extract and aloe vera, allantoin, emollients, humectants (e.g., glycerin), disinfectants, hydroxy acids for wrinkle control or anti-aging effects, sunscreens, tanning promoters, skin lighteners, deodorants and anti-perspirants, ceramides for skin benefits and other uses, astringents, moisturizers, nail polish removers, insect repellants, antioxidants, antiseptics, anti-inflammatory agents and the like.

Suitable odor control additives for use in the wetting composition and pre-moistened wipes of the present invention include, but are not limited to, zinc salts; talc powder; encapsulated perfumes (including microcapsules, macrocapsules, and perfume encapsulated in liposomes, vessicles, or microemulsions); chelants, such as ethylenediamine tetra-acetic acid; zeolites; activated silica, activated carbon granules or fibers; activated silica particulates; polycarboxylic acids, such as citric acid; cyclodextrins and cyclodextrin derivatives; chitosan or chitin and derivatives thereof; oxidizing agents; antimicrobial agents, including silver-loaded zeolites; kieselguhr; and mixtures thereof. In addition to controlling odor from the body or body wastes, odor control strategies can also be employed to mask or control any odor of the treated substrate. Typically, the wetting composition contains less than about 5 weight percent of odor control additives based on the total weight of the wetting composition. In another aspect, the wetting composition contains from about 0.01 weight percent to about 2 weight percent of odor control additives. Yet, in another aspect, the wetting composition contains from about 0.03 weight percent to about 1 weight percent of odor control additives.

The wetting composition and/or pre-moistened wipes may include derivatized cyclodextrins, such as hydroxypropyl beta-cyclodextrin in solution, which remain on the skin after wiping and provide an odor-absorbing layer. In other embodiments, the odor source is removed or neutralized by application of an odor-control additive, exemplified by the action of a chelant that binds metal groups necessary for the function of many proteases and other enzymes that commonly produce an odor. Chelating the metal group interferes with the enzyme's action and decreases the risk of malodor in the product.

The wetting composition of the present invention can be further modified by the addition of solid particulates or microparticulates. Suitable particulates include, but are not limited to, mica, silica, alumina, calcium carbonate, kaolin, talc, and zeolites. The particulates can be treated with stearic acid or other additives to enhance the attraction or bridging of the particulates to the binder system, if desired. Also, two-component microparticulate systems, commonly used as retention aids in the papermaking industry, can be used. Such two-component microparticulate systems generally comprise a colloidal particle phase, such as silica particles, and a water-soluble cationic polymer for bridging the particles to the fibers of the web to be formed. The presence of particulates in the wetting composition can serve one or more useful functions, such as (1) increasing the opacity of the pre-moistened wipes; (2) modifying the rheology or reducing the tackiness of the pre-moistened wipe; (3) improving the tactile properties of the wipe; or (4) delivering desired agents to the skin via a particulate carrier, such as a porous carrier or a microcapsule. Typically, the wetting composition contains less than about 25 weight percent of particulate based on the total weight of the wetting composition. In another aspect, the wetting composition contains from about 0.05 weight percent to about 10 weight percent of microparticulate. Yet, in another aspect, the wetting composition contains from about 0.1 weight percent to about 5 weight percent of microparticulate.

Microcapsules and other delivery vehicles can also be used in the wetting composition of the present invention to provide skin-care agents; medications; comfort promoting agents, such as eucalyptus; perfumes; skin care agents; odor control additives; vitamins; powders; and other additives to the skin of the user. For example, the wetting composition can contain up to about 25 weight percent of microcapsules or other delivery vehicles based on the total weight of the wetting composition. In another aspect, the wetting composition can contain from about 0.05 weight percent to about 10 weight percent of microcapsules or other delivery vehicles. Yet, in another aspect, the wetting composition can contain from about 0.2 weight percent to about 5.0 weight percent of microcapsules or other delivery vehicles.

Microcapsules and other delivery vehicles are well known in the art. Known additives, including but not limited to benzoyl peroxide, salicylic acid, retinol, retinyl palmitate, octyl methoxycinnamate, tocopherol, silicone compounds, and mineral oil, can be used with delivery agents comprising e.g., soft, hollow spheres that can contain an additive at over 10 times the weight of the delivery vehicle; sponge-like materials (optionally used with silicone and mineral oil); cyclodextrins and their derivatives; liposomes; polymeric sponges; and spray-dried starch. Additives present in microcapsules can be isolated from the environment and the other agents in the wetting composition until the wipe is applied to the skin, whereupon the microcapsules break and deliver their load to the skin or other surfaces.

The wetting composition of the present invention can contain preservatives and/or anti-microbial agents. Several preservatives and/or anti-microbial agents useful in the present invention include, but are not limited to, phenoxyethanol, Mackstat H 66 (available from McIntyre Group, Chicago, Ill.), DMDM hydantoin (e.g., Glydant Plus™, Lonza, Inc., Fair Lawn, N.J.), iodopropynyl butylcarbamate, Kathon (Rohm and Hass, Philadelphia, Pa.), methylparaben, propylparaben, 2-bromo-2-nitropropane-1,3-diol, benzoic acid, and the like. Typically, the wetting composition contains less than about 2 weight percent on an active basis of preservatives and/or antimicrobial agents based on the total weight of the wetting composition. In another aspect, the wetting composition contains from about 0.01 weight percent to about 1 weight percent of preservatives and/or anti-microbial agents. Yet, in another aspect, the wetting composition contains from about 0.01 weight percent to about 0.5 weight percent of preservatives and/or anti-microbial agents.

A variety of wetting agents and/or cleaning agents can be used in the wetting composition of the present invention. Suitable wetting agents and/or cleaning agents include, but are not limited to, detergents and nonionic, amphoteric, and anionic surfactants, especially amino acid-based surfactants. Amino acid-based surfactant systems, such as those derived from amino acids L-glutamic acid and other natural fatty acids, offer pH compatibility to human skin and good cleansing power, while being relatively safe and providing improved tactile and moisturization properties compared to other anionic surfactants. One function of the surfactant is to improve wetting of the dry substrate with the wetting composition. Another function of the surfactant can be to disperse bathroom soils when the pre-moistened wipe contacts a soiled area and to enhance their absorption into the substrate. The surfactant can further assist in make-up removal, general personal cleansing, hard surface cleansing, odor control, and the like. Typically, the wetting composition contains less than about 3 weight percent of wetting agents and/or cleaning agents based on the total weight of the wetting composition. In another aspect, the wetting composition contains from about 0.01 weight percent to about 2 weight percent of wetting agents and/or cleaning agents. Yet, in another aspect, the wetting composition contains from about 0.1 weight percent to about 0.5 weight percent of wetting agents and/or cleaning agents.

In addition to amino-acid based surfactants, a wide variety of surfactants can be used in the present invention. Suitable non-ionic surfactants include, but are not limited to, the condensation products of ethylene oxide with a hydrophobic (oleophilic) polyoxyalkylene base formed by the condensation of propylene oxide with propylene glycol. The hydrophobic portion of these compounds desirably has a molecular weight sufficiently high so as to render it water-insoluble. The addition of polyoxyethylene moieties to this hydrophobic portion increases the water-solubility of the molecule as a whole, and the liquid character of the product is retained up to the point where the polyoxyethylene content is about 50% of the total weight of the condensation product, such as those in which the polyoxypropylene ether has a molecular weight of about 1500-3000 and the polyoxyethylene content is about 35-55% of the molecule by weight.

Other useful nonionic surfactants include, but are not limited to, the condensation products of C₈-C₂₂ alkyl alcohols with 2-50 moles of ethylene oxide per mole of alcohol. Other nonionic surfactants, which can be employed in the wetting composition of the present invention, include the ethylene oxide esters of C₆-C₁₂ alkyl phenols such as (nonylphenoxy)polyoxyethylene ether (e.g., esters prepared by condensing about 8-12 moles of ethylene oxide with nonylphenol). Further non-ionic surface active agents include, but are not limited to, alkyl polyglycosides (APG), derived as a condensation product of dextrose (D-glucose) and a straight or branched chain alcohol. The glycoside portion of the surfactant provides a hydrophile having high hydroxyl density, which enhances water solubility. Additionally, the inherent stability of the acetal linkage of the glycoside provides chemical stability in alkaline systems. Furthermore, unlike some non-ionic surface active agents, alkyl polyglycosides have no cloud point, allowing one to formulate without a hydrotrope, and these are very mild, as well as readily biodegradable non-ionic surfactants.

Silicones are another class of wetting agents available in pure form, or as microemulsions, macroemulsions, and the like. One exemplary non-ionic surfactant group is the silicone-glycol copolymers. These surfactants are prepared by adding poly(lower)alkylenoxy chains to the free hydroxyl groups of dimethylpolysiloxanols. These surfactants function, with or without any volatile silicones used as solvents, to control foaming produced by the other surfactants, and also impart a shine to metallic, ceramic, and glass surfaces.

Anionic surfactants can be used in the wetting compositions of the present invention. Anionic surfactants that are useful due to their high detergency include anionic detergent salts having alkyl substituents of 8 to 22 carbon atoms such as the water-soluble higher fatty acid alkali metal soaps, e.g., sodium myristate and sodium palmitate. A class of anionic surfactants which can be employed in the invention include, but are not limited to, the water-soluble sulfated and sulfonated anionic alkali metal and alkaline earth metal detergent salts containing a hydrophobic higher alkyl moiety (typically containing from about 8 to 22 carbon atoms) such as salts of higher alkyl mono or polynuclear aryl sulfonates having from about 1 to 16 carbon atoms in the alkyl group.

Other classes of anionic surfactants which can be used with the invention include, but are not limited to, the alkali metal salts of alkyl naphthalene sulfonic acids (methyl naphthalene sodium sulfonate); sulfated higher fatty acid monoglycerides such as the sodium salt of the sulfated monoglyceride of cocoa oil fatty acids and the potassium salt of the sulfated monoglyceride of tallow fatty acids; alkali metal salts of sulfated fatty alcohols containing from about 10 to 18 carbon atoms (e.g., sodium lauryl sulfate and sodium stearyl sulfate); sodium C₁₄-C₁₆-alphaolefin sulfonates; alkali metal salts of sulfated ethyleneoxy fatty alcohols (the sodium or ammonium sulfates of the condensation products of about 3 moles of ethylene oxide with a C₁₂-C₁₅ n-alkanol; alkali metal salts of higher fatty esters of low molecular weight alkylol sulfonic acids, e.g., fatty acid esters of the sodium salt of isothionic acid, the fatty ethanolamide sulfates; the fatty acid amides of amino alkyl sulfonic acids, e.g., lauric acid amide of taurine; as well as numerous other anionic organic surface active agents such as sodium xylene sulfonate, sodium naphthalene sulfonate, sodium toulene sulfonate and mixtures thereof.

A further useful class of anionic surfactants includes the 8-(4-n-alkyl-2-cyclohexenyl)-octanoic acids, wherein the cyclohexenyl ring is substituted with an additional carboxylic acid group. In general, these anionic surface active agents can be employed in the form of their alkali metal salts, ammonium or alkaline earth metal salts.

The wetting composition can further comprise an aqueous microemulsion of silicone particles, for example, organopolysiloxanes in an aqueous microemulsion. The wetting composition can include less than about 5 weight percent of a microemulsion of silicone particles based on the total weight of the wetting composition. In another aspect, the wetting composition contains from about 0.02 weight percent to about 3 weight percent of a microemulsion of silicone particles. Yet in another aspect, the wetting composition contains from about 0.02 weight percent to about 0.5 weight percent of a microemulsion of silicone particles.

Silicone emulsions in general can be applied to the pre-moistened wipe by any known coating method. For example, the pre-moistened wipe may be moistened with a wetting composition comprising a water-dispersible or water-miscible, silicone-based component. In one aspect of the present invention, the wetting composition comprises a silicone copolyol sulfosuccinate, such as disodium dimethicone copolyol sulfosuccinate and diammonium dimethicone copolyolsulfosuccinate. In another aspect, the wetting composition comprises less than about 2 percent by weight of the silicone-based sulfosuccinate, and, in another aspect, from about 0.05 percent to about 0.30 percent by weight of the silicone-based sulfosuccinate.

In another example of a product comprising silicone emulsion, a powder can be present in the wetting composition for controlling skin oils, which powder can be spherical and contain a dimethicone/vinyldimethicone cross-polymer. Thus, a water dispersible wipe that delivers a powder effective in controlling skin oil, is also within the scope of the present invention.

The wetting composition of the present invention can contain one or more emollients or humectants (e.g., glycerin). Suitable emollients include, but are not limited to, PEG 75 lanolin, methyl gluceth 20 benzoate, C₁₂-C₁₅ alkyl benzoate, ethoxylated cetyl stearyl alcohol, products marketed as Lambent wax WS-L, Lambent WD-F, Cetiol HE (Henkel Corp.), Glucam P20 (Amerchol), Polyox WSR N-10 (Union Carbide), Polyox WSR N-3000 (Union Carbide), Luviquat (BASF), Finsolv SLB 101 (Finetex Corp.), mink oil, allantoin, stearyl alcohol, Estol 1517 (Unichema), Finsolv SLB 201 (Finetex Corp.), or any combination thereof.

An emollient can also be applied to a surface of the web prior to or after wetting with the wetting composition. Such an emollient can be insoluble in the wetting composition and can be immobile except when exposed to a force. For example, a petrolatum-based emollient can be applied to one surface in a pattern, after which the other surface is wetted to saturate the wipe. Such a product could provide a cleaning surface and an opposing skin treatment surface.

The emollient composition in such products and other products of the present invention can comprise a plastic or fluid emollient such as one or more liquid hydrocarbons (e.g., petrolatum), mineral oil and the like, vegetable and animal fats (e.g., lanolin, phospholipids and their derivatives) and/or a silicone materials such as one or more alkyl substituted polysiloxane polymers, including the polysiloxane emollients. Optionally, a hydrophilic surfactant can be combined with a plastic emollient to improve wettability of the coated surface. Liquid hydrocarbon emollients and/or alkyl substituted polysiloxane polymers may be blended or combined with one or more fatty acid ester emollients derived from fatty acids or fatty alcohols.

The emollient material may be in the form of an emollient blend. For example, the emollient blend can comprise a combination of one or more liquid hydrocarbons (e.g., petrolatum), mineral oil and the like, vegetable and animal fats (e.g., lanolin, phospholipids and their derivatives), with a silicone material such as one or more alkyl substituted polysiloxane polymers. In another aspect, the emollient blend comprises a combination of liquid hydrocarbons (e.g., petrolatum) with dimethicone or with dimethicone and other alkyl substituted polysiloxane polymers.

Water-soluble, self-emulsifying emollient oils, which can be used in the present wetting compositions, include the polyoxyalkoxylated lanolins and the polyoxyalkoxylated fatty alcohols. The polyoxyalkoxy chains comprise mixed propylenoxy and ethyleneoxy units. The lanolin derivatives typically comprise about 20-70 such lower-alkoxy units while the C₁₂-C₂₀-fatty alcohols will be derivatized with about 8-15 lower-alkyl units. A non-limiting example of such a lanolin derivative is Lanexol AWS (PPG-12-PEG-50, Croda, Inc., New York, N.Y.). A non-limiting example of a poly(15-20)C₂-C₃-alkoxylate is PPG-5-Ceteth-20, known as Procetyl AWS (Croda, Inc.).

Surface-feel modifiers can be employed to improve the tactile sensation (e.g., lubricity) of the skin during use of the product. Suitable surface feel modifiers include, but are not limited to, commercial debonders; and softeners, such as the softeners used in the art of tissue making including quaternary ammonium compounds with fatty acid side groups, silicones, waxes, and the like. Typically, the wetting composition contains less than about 2 weight percent of surface feel modifiers based on the total weight of the wetting composition. In another aspect, the wetting composition contains from about 0.01 weight percent to about 1 weight percent of surface feel modifiers. Yet, in another aspect, the wetting composition contains from about 0.01 weight percent to about 0.05 weight percent of surface feel modifiers.

A variety of fragrances can be used in the wetting composition of the present invention. Typically, the wetting composition contains less than about 2 weight percent of fragrances based on the total weight of the wetting composition. In another aspect, the wetting composition contains from about 0.01 weight percent to about 1 weight percent of fragrances. Yet, in another aspect, the wetting composition contains from about 0.01 weight percent to about 0.05 weight percent of fragrances.

Further, a variety of fragrance solubilizers can be used in the wetting composition of the present invention. Suitable fragrance solubilizers include, but are not limited to, polysorbate 20, propylene glycol, ethanol, isopropanol, diethylene glycol monoethyl ether, dipropylene glycol, diethyl phthalate, triethyl citrate, and the like.

Opacifers can be employed in the wetting composition. Suitable opacifiers include, but are not limited to, titanium dioxide or other minerals or pigments, and synthetic opacifiers.

The water dispersible wet wipe of the present invention can be incorporated into a variety of products. Non-limiting examples of products include personal care wipers, household cleaning wipers and wipers for other hard surface environments such as restaurants and hospitals, disinfecting wipes and dusting wipers. The wipers may disinfect hard surfaces.

Personal care wipers can be impregnated with, e.g., emollients, humectants, fragrances, and the like. Household cleaning wipers or hard surface cleaning wipers can be impregnated with, e.g., surfactants (for example, quaternary amines), peroxides, solvents, chelating agents, antimicrobials, fragrances, and the like. Dusting wipers can be impregnated with, e.g., oils.

Various types of wipers include baby wipes, cosmetic wipes, disposable washcloths, household cleaning wipes, such as kitchen wipes, bath wipes, or hard surface wipes (for e.g., restaurant and hospital environments), disinfecting and germ removal wipes, specialty cleaning wipes, such as glass wipes, mirror wipes, leather wipes, electronics wipes, lens wipes, or polishing wipes, medical cleaning wipes, dusting wipes, and the like.

### EXAMPLES

### Example 1

Handsheets were prepared using two types of CMF prepared from Lyocell dull grade and fast-fibrillating Lyocell. Both fiber types are available from Lenzing AG, Lenzing, Austria. The cellulose microfibers from fast-fibrillating Lyocell were fibrillated in a pulper using less than half the energy used for Lyocell dull grade to reach an end point. The Lyocell dull grade was fibrillated in a disk refiner. The fiber lengths of the fast-fibrillating Lyocell CMF remained longer than that made from Lyocell dull grade.

Wet tensile tests were performed to simulate wiper conditions. Wet tensile was tested on one inch strips wetted with either standard water or a wetting composition including the following ingredients: water, a surface feel modifier, three antimicrobial agents, two botanicals, a vitamin, an acid, and fragrance. The pH of the wetting composition was 5.0±0.5. The strips were wetted to approximately 25% solids to simulate wet wiper conditions. Wet tensile was tested at 0 minutes, 10 minutes, and 24 hours. Strips prepared for 10 minutes and 24-hour tests were stored in a plastic bag to prevent evaporation.

Dispersibility of the handsheets was tested in accordance with EDANA/INDA FG502 guidelines (the “Slosh Box” test), a wiper test, with some modifications. Samples were agitated for three hours in two liters of tap water. The percent by weight of the handsheet that did not pass through a sieve with half-inch holes was measured.

Dispersibility of the handsheets and basesheets was also measured with the wiper “Slosh Box” test, with two modifications. A handsheet or basesheet was sloshed in water until it broke into pieces less than 1 inch in diameter. The time was recorded to the nearest 0.5 minutes rather than waiting the full three hours. Each test was performed in triplicate. In some handsheets and basesheets, a glyoxalated polyacrylamide wet strength agent was used.

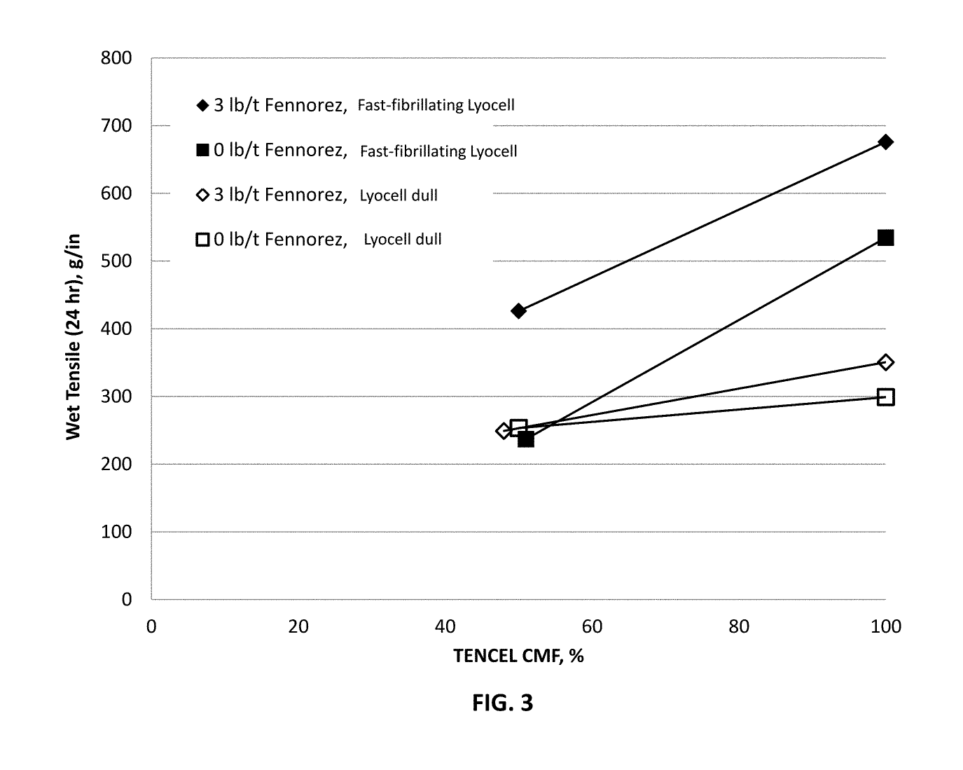

FIG. 3 is a graph illustrating wet tensile of handsheets wetted for 24 hours as a function of % cellulose microfibers. As shown, cellulose microfiber alone provided sufficiently high wet tensile, without the addition of the wet strength resin. The Fast-fibrillating Lyocell regenerated cellulose microfibers (solid symbols) made stronger wet handsheets than commercially available Lyocell dull cellulose microfibers (open symbols). Even after 24 hours, a residual level of temporary wet strength persisted.

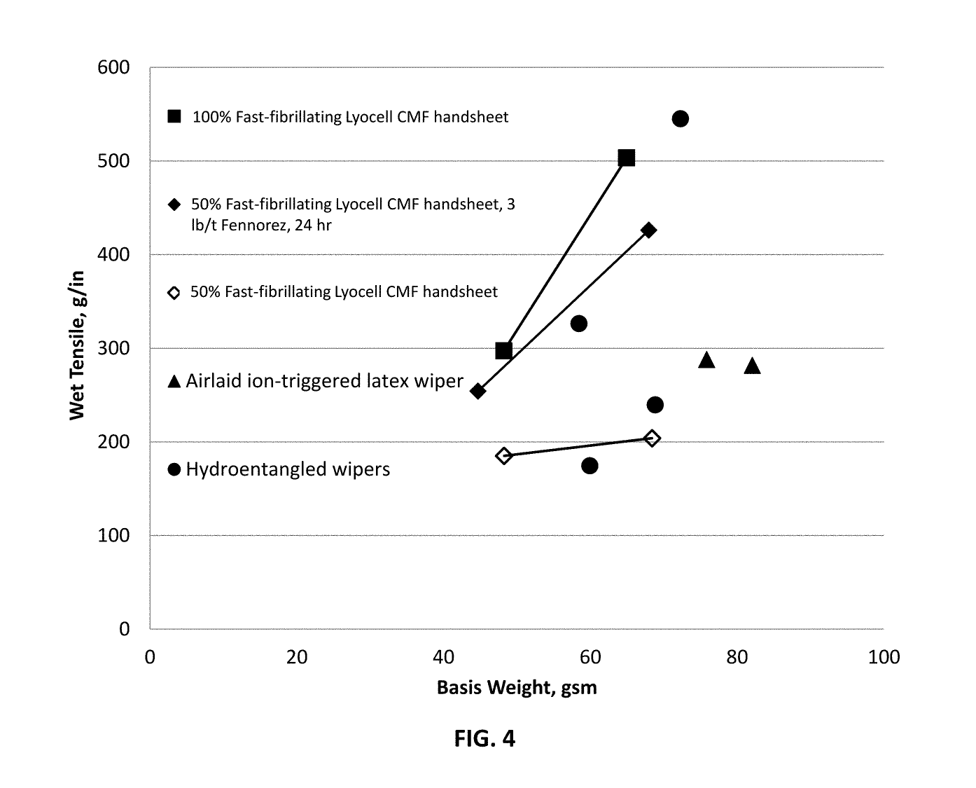

FIG. 4 is a graph comparing wet tensile of handsheets. Existing market wipes (hydroentangled and airlaid ion-triggered latex) had wet tensile of about 200-300 g/in. In contrast, handsheets prepared with the Fast-fibrillating Lyocell CMF had improved wet tensile even without wet strength agents, additives, or hydroentangling.

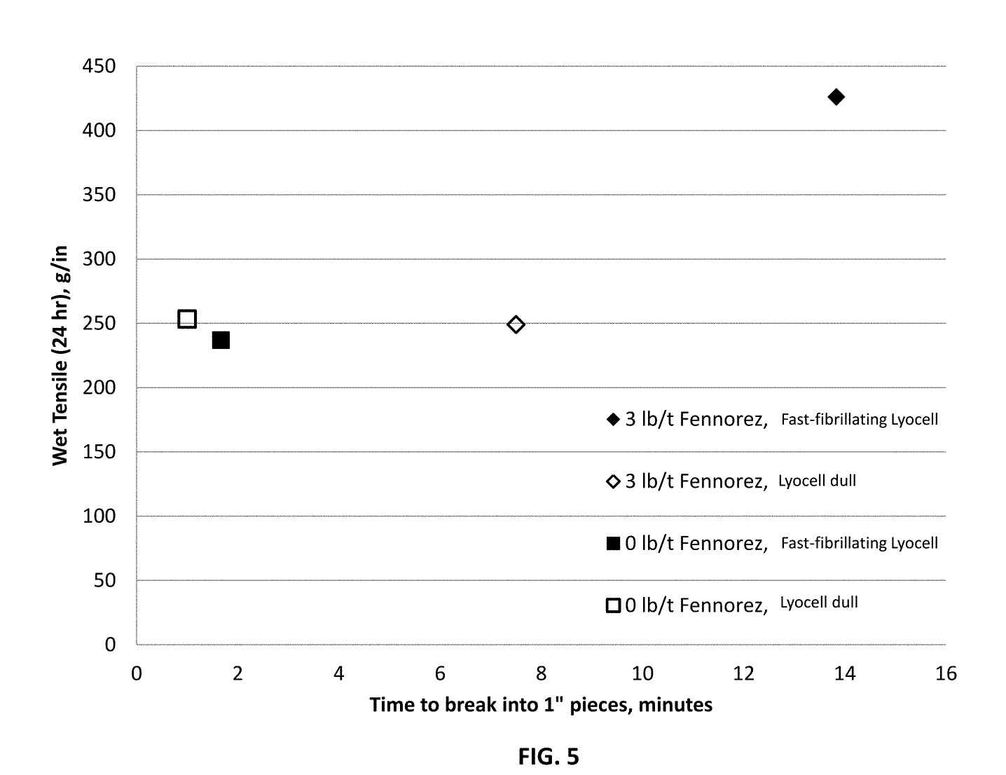

FIG. 5 is a graph showing dispersibility of the 60 gsm handsheets using the Slosh Box test. Some handsheets included the glyoxalated polyacrylamide wet strength agent and all had a 50/50 blend of cellulose microfibers and northern softwood Kraft wood pulp fibers. Handsheets including 50% of the fibrillated regenerated cellulose microfibers (“Fast-fibrillating Lyocell”) were compared to handsheets including 50% of CMF from commercially available Lyocell (“Lyocell dull”). All handsheets broke down into pieces of less than one inch in less than 15 minutes.

### Example 2

Handsheets were prepared with a combination of refined northern softwood fibers, CMF, and a temporary wet strength resin. Glyoxalated polyacrylamide (GPAM) was used as the wet strength agent. GPAM was added to the fibers in an amount of about 3 lb/t.

Wet tensile of the handsheets was tested on one inch strips wetted with the wetting composition described above for Example 1. The target pH was 5.0±0.5. An alternative wetting composition was tested with a pH of about 8 (adjusted with sodium bicarbonate).

The strips were wetted to approximately 25% solids. Wet tensile was tested at 10 minutes, 24 hours, and 7 days. Testing performed at 7 days under two sets of conditions: (1) room temperature, and (2) 100° F. with 90% relative humidity. The second set of conditions was used to accelerate aging over time. For example, 7 days at 100° F. and 90% relative humidity could simulate as long as 4 weeks in ambient conditions. The strips were stored in sealed plastic bags to prevent evaporation. For strips aged at 100° F., the strips were placed in sealed plastic bags and then the bags were stacked and wrapped in aluminum foil to further retard evaporation at elevated temperature.

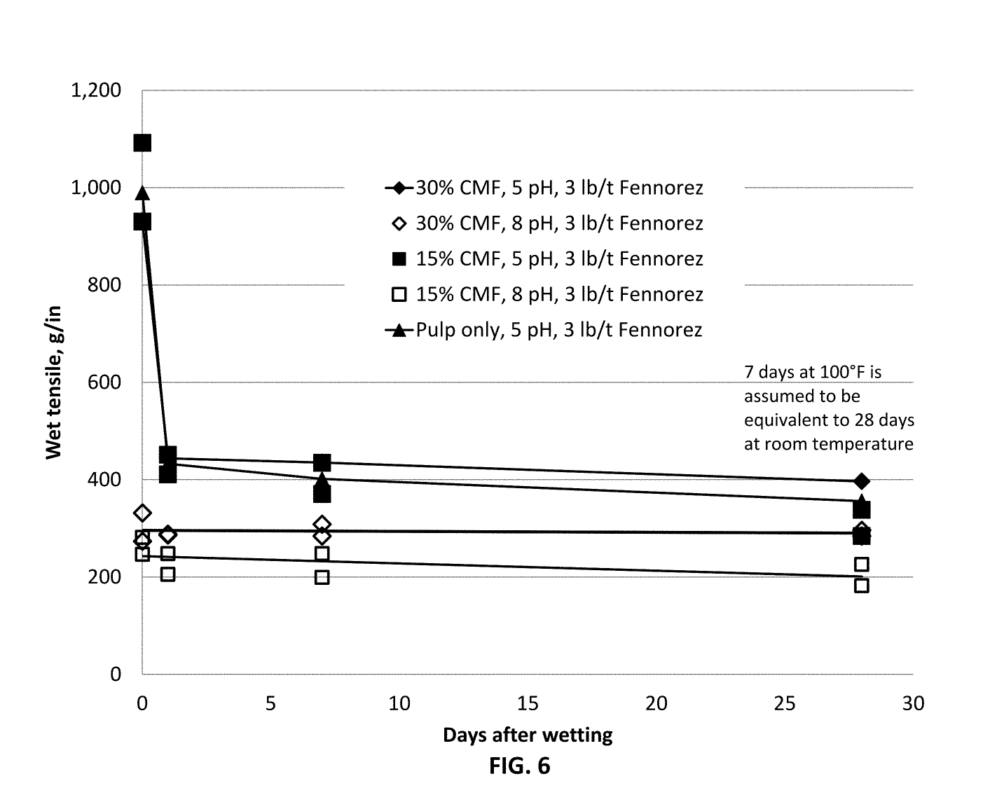

FIG. 6 is a graph illustrating wet tensile decay of the wetted handsheets over time. The wet tensile in the sheets with the temporary wet strength agent initially decayed rapidly. The wipers stored in a wetting composition having a pH of 5 retained wet tensile after the initial decay. In contrast, the wipers stored in the wetting composition with a pH of 8 had substantially lower wet tensile.

### Example 3

A basesheet of about 28 gsm was made on a tissue machine with furnish including 80% Northern Bleached Softwood Kraft (NB SK) pulp and 20% CMF made from fast-fibrillating Lyocell. About 4 lb/T GPAM was added to the furnish. The physical properties of the dry basesheet are shown in Table 1. The CD wet tensile of the basesheet was measured immediately after wetting with standard water (deionized water with hardness and neutral pH, to simulate conventional tap water).

TABLE 1
Basesheet properties

| | | Caliper | | | | | | Wet Tensile | |
|---|---|---|---|---|---|---|---|---|---|
| | | 8 sheet | Basis | Tensile | Stretch | Tensile | Stretch | (Finch Cup) | Tensile |
| | GPAM, | mils/8 | weight | MD | MD | CD | CD | CD | GM |
| Furnish | lb/T | sheet | gsm | g/3 in | % | g/3 in | % | g/3 in | g/3 in |
| 80% NBSK/ | 4.1 | 90.2 | 28.8 | 3,502 | 19.7 | 3,927 | 6.0 | 1,011 | 3,707 |
| 20% CMF | | | | | | | | | |
| 80% NBSK/ | 4.1 | 93.8 | 29.3 | 3,660 | 20.2 | 4,101 | 6.5 | 1,097 | 3,873 |
| 20% CMF | | | | | | | | | |

The basesheet was converted into a two-ply product. The dry properties of the two-ply products are shown in Table 2. The wet tensile of the dry wiper was measured immediately after wetting with standard water.

TABLE 2
Dry wiper properties

| | Caliper | | | Wet Tens | | | |
|---|---|---|---|---|---|---|---|
| Basis | 8 Sheet | Tensile | Tensile | (Finch Cup) | Tensile | Stretch | Stretch |
| Weight | mils/8 | MD | CD | CD | GM | MD | CD |
| gsm | sht | g/3 in | g/3 in | g/3 in | g/3 in | % | % |
| 58.3 | 160 | 8,011 | 7,034 | 1,851 | 7,505 | 19.5 | 6.7 |
| 58.9 | 166 | 7,542 | 7,211 | 1,863 | 7,372 | 19.1 | 6.1 |

The dry wiper was loaded with a pH-modifying lotion and allowed to come to equilibrium before testing. The properties of the wetted wiper are shown in Table 3. The Slosh Box test demonstrated 90% dispersibility after three hours.

TABLE 3
Wet wipe properties

| | | Tensile | Tensile | Stretch | Stretch | TEA | TEA | | Slosh |
|---|---|---|---|---|---|---|---|---|---|
| | | Wiper | Wiper | Wiper | Wiper | Wiper | Wiper | Caliper | Box |
| | Basis | 1 × 4″ | 1 × 4″ | 1 × 4″ | 1 × 4″ | 1 × 4″ | 1 × 4″ | Wiper | % |
| Moisture | Weight | MD | CD | MD | CD | MD | CD | mils/ | Disint. |
| % | gsm | g/in | g/in | % | % | g/mm | g/mm | sheet | % |
| 71.4 | 59.8 | 247 | 212 | 15.6 | 7.5 | 0.67 | 0.35 | 12.4 | 90 |

### Example 4

A basesheet of about 17 gsm was made on a tissue machine with furnish including different ratios of Northern Bleached Softwood Kraft (NB SK) pulp and CMF made from fast-fibrillating Lyocell. A glyoxalated polyacrylamide wet strength agent (FJ98) was added to the furnish. The physical properties of the dry basesheet are shown in Table 4 below.

The CD tensile of the basesheets was lower than the MD tensile. Likewise, the ratio of MD tensile to CD tensile increased, compared to, for example, the basesheets in Example 3 (Table 1). The basesheets in Example 3 (Table 1) had a CD tensile that was greater than the MD tensile. Although generally, a ratio of MD tensile to CD tensile is desired to be about 1:1 (as in Example 3, Table 1), the basesheets in Table 4 have a ratio of MD tensile to CD tensile of about 2.5:1. A lower CD tensile means that the basesheet will a lower overall strength, and therefore, have an increased water dispersibility.

The amount of wet strength agent (GPAM) was also less than that used in the basesheets in Example 3, Table 1 (1.5 to 2.5 lb/T compared to 4.1 lb/T). Because the basesheets had a lower overall strength, due to the decreased CD tensile, less wet strength agent could be used. Using less wet strength agent also results in increased water dispersibility.

The MD and CD wet tensile of the basesheet was measured immediately after wetting with standard water (deionized water with hardness and neutral pH, to simulate conventional tap water). The MD wet tensile facilitates converting of the web after wetting.

TABLE 4
Basesheet properties

| | | Caliper | | | | | | Wet Tensile | Wet Tensile | |
|---|---|---|---|---|---|---|---|---|---|---|
| | | 8 sheet | Basis | Tensile | Stretch | Tensile | Stretch | (Finch Cup) | (Finch Cup) | Tensile |
| | GPAM | mils/8 | weight | MD | MD | CD | CD | MD | CD | GM |
| Furnish | lb/T | sheet | gsm | g/3 in | % | g/3 in | % | g/3 in | g/3 in | g/3 in |
| 71% NBSK/ | 2.5 | 116.4 | 17.1 | 5185 | 18.4 | 1826 | 7.1 | 1,452.73 | 521.0 | 3077 |
| 29% CMF | | | | | | | | | | |
| 70% NBSK/ | 2.0 | 107.5 | 17.0 | 4327 | 18.9 | 1673 | 8.7 | 1,047.80 | 330.0 | 2690 |
| 30% CMF | | | | | | | | | | |
| 71% NBSK/ | 1.5 | 101.3 | 17.0 | 4069 | 19.0 | 1719 | 9.1 | 778.96 | 312.1 | 2645 |
| 29% CMF | | | | | | | | | | |
| 79% NBSK/ | 2.5 | 108.8 | 17.5 | 4666 | 18.6 | 1816 | 9.7 | 1,145.55 | 347.6 | 2911 |
| 21% CMF | | | | | | | | | | |
| 80% NBSK/ | 2.0 | 118.1 | 16.6 | 4743 | 18.8 | 1985 | 9.7 | 908.16 | 383.9 | 3069 |
| 20% CMF | | | | | | | | | | |

The basesheets were loaded with a pH-modifying lotion and allowed to come to equilibrium before testing. The properties of the wetted sheets are shown in Table 5. The Slosh Box test was performed in triplicate and demonstrated up to 93% dispersibility after 20 minutes. All measurements were conducted on two plies of basesheet to simulate a two-ply wipe.

TABLE 5
Wetted basesheets

| | | | | Tensile | Tensile | Stretch | Stretch | | Slosh |
|---|---|---|---|---|---|---|---|---|---|
| | | | | Wiper | Wiper | Wiper | Wiper | Caliper | Box |
| | | | Basis | 1 × 4″ | 1 × 4″ | 1 × 4″ | 1 × 4″ | Wiper | % |
| | GPAM | Moisture | Weight | MD | CD | MD | CD | mils/ | Disint |
| Furnish | lb/T | % | gsm | g/in | g/in | % | % | sheet | 20 mins |
| 71% NBSK/ | 2.5 | 68.14 | 60.73 | 413.1 | 152.0 | 14.7 | 10.7 | 11.8 | 5.7 |
| 29% CMF | | | | | | | | | |
| 70% NBSK/ | 2.0 | 67.73 | 58.78 | 337.0 | 113.7 | 15.1 | 11.7 | 11.9 | 84.2 |
| 30% CMF | | | | | | | | | |
| 71% NBSK/ | 1.5 | 68.12 | 59.45 | 282.1 | 110.4 | 14.9 | 12.1 | 9.7 | 92.9 |
| 29% CMF | | | | | | | | | |

### Example 5

Basesheets were made on a tissue machine. Basesheets with pulp only (NBSK) were compared to basesheets with mixtures of pulp and 20% or 30% CMF made with fast-fibrillating Lyocell. Increasing amounts of wet strength agent (a glyoxalated polyacrylamide) (1.5-3.0 lb/T) was added to the furnishes.

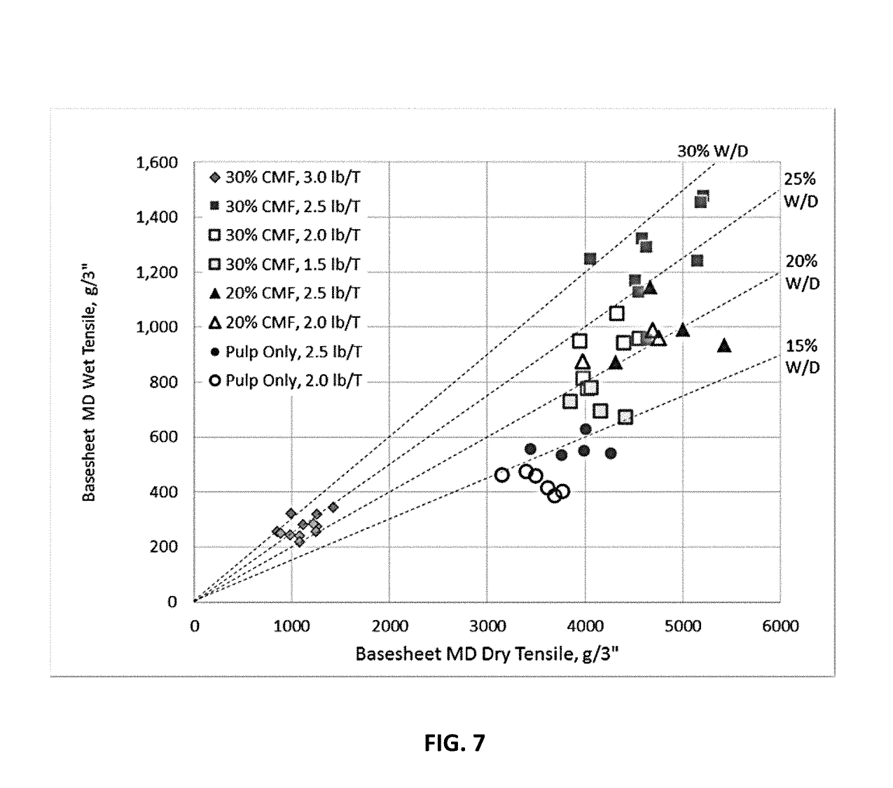

The MD wet tensile was measured immediately after wetting with standard water (deionized water with hardness and neutral pH, to simulate conventional tap water). FIG. 7 is a graph showing the MD wet tensile as a function of the MD dry tensile. As shown in FIG. 7, basesheets with CMF had a higher ratio of wet to dry (W/D) strength than basesheets with pulp only. Increasing the amount of wet strength agent (lb/T) also increased the MD wet tensile strength. At a given dose of temporary wet strength, a higher MD wet tensile was obtained compared to pulp only.

### Example 6

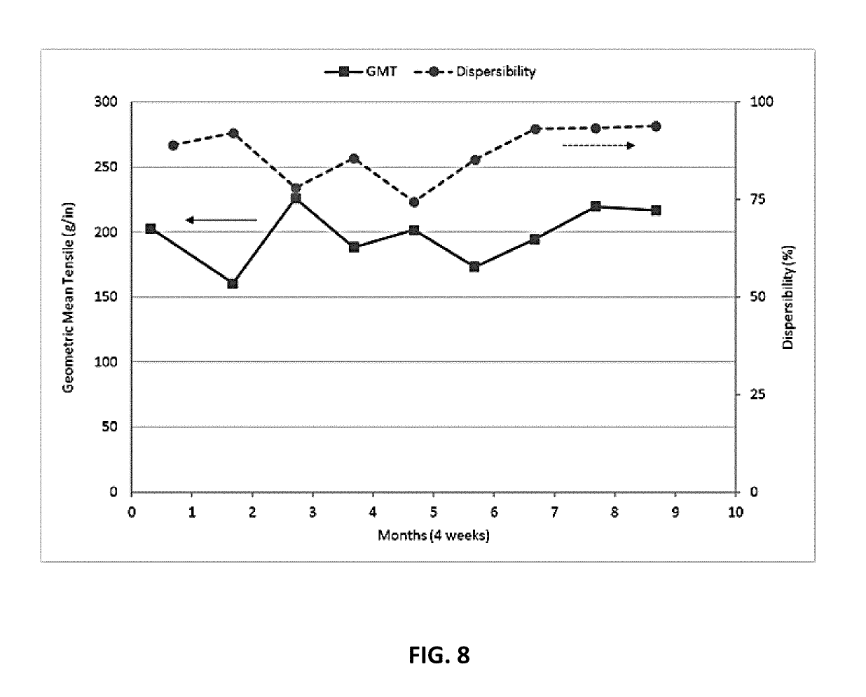

Converted, packaged wipes were pre-moistened with a pH modifying lotion at 220% liquid per dry weight of the wipe. GM Tensile and Slosh Box dispersibility were measured over time to assess the shelf stability of the wipe wet. FIG. 8 is a graph illustrating long-term shelf stability of the wet wipes per four week interval (month). The solid line represents the GM tensile strength, and the dashed line represents Slosh Box dispersibility after three hours. As shown, over about 8 months, neither the geometric mean wet tensile strength (g/in) nor the dispersibility significantly decreased over time.

According to one or more aspects, a water dispersible wipe includes a web including at least 10 weight % (wt. %) of a fibrillated regenerated cellulose microfiber (CMF) based on the total weight of the web, the remaining fibers of the web comprising natural cellulosic fibers; a wet strength agent disposed within the web; and a pH-modifying composition having a wetting pH disposed within the web; wherein the water dispersible wipe comprises at least two webs and disperses upon exposure to an aqueous environment having a pH greater than the wetting pH and has a cross-direction (CD) wet tensile of at least 75 g/in after 24 hours as measured according to IVDA Standard Test WSP 110.4 (05).

In another aspect, the CD wet tensile is at least 100 g/in after 24 hours.

According to some aspects, the pH-modifying composition is malic acid, citric acid, hydrochloric acid, sulfuric acid, or any combination thereof.

In other aspects, the pH-modifying composition is a wetting composition.

The wetting composition can include an additive, and the additive is a skin-care additive, an odor control agent, a disinfectant, a particulate, an antimicrobial agent, a preservative, a wetting agent, a cleaning agent, an emollient, a surface feel modifier, a fragrance, a fragrance solubilizer, an opacifier, a fluorescent whitening agent, an ultraviolet light absorber, or any combination thereof.

Yet, in other aspects, the wetting pH is in a range from about 3.5 to about 6.5.

Still yet, in other aspects, the web before plying has a basis weight in a range from about 20 to about 60 grams per square meter (gsm).

In one or more aspects, the wet strength agent is a glyoxalated wet strength agent.

In some aspects, the natural cellulosic fibers are softwood fibers, hardwood fibers, or a combination thereof.

In other aspects, the wetting composition has a pH in a range from about 4.5 to about 5.5.

Yet, in other aspects, the natural cellulosic fibers are non-wood fibers. The non-wood fibers are bast fibers in some aspects. The non-wood fibers are flax fibers, hemp fibers, jute fibers, ramie fibers, nettle fibers, Spanish broom fibers, kenaf plant fibers, arundo donax fibers, or any combination thereof in other aspects.

Still yet, in some aspects, the CMF has a length-weighted average length after fibrillation in a range from about 1.5 to about 5.0 millimeters (mm).

According to one or more aspects, the web comprises the CMF in an amount in a range from about 10 wt. % to about 50 wt. %.

In some aspects, the wet strength agent is present in an amount of about 0.01 to about 8 pounds per ton (lb/t).

In other aspects, the water dispersible wipe is a wet wipe.

Still yet, in other aspects, the water dispersible wipe is a dry wipe.

In some aspects, the water dispersible wipe has a CD wet tensile in a range from about 75 to about 500 Win after 24 hours.

According to one or more aspects, a water dispersible wet wipe includes a web including a CMF in a range from about 10 wt. % to about 100 wt. % based on the total weight of the web, the remaining fibers of the web including natural cellulosic fibers, and the CMF having a freeness of less than 175 milliliters (mL); a glyoxalated wet strength agent disposed within the web; and a pH-modifying composition having a wetting pH disposed within the web; wherein the water dispersible wet wipe disperses upon exposure to an aqueous environment having a pH greater than the wetting pH and has a CD wet tensile of at least 200 grams/3 inch (g/3 in) as measured according to TAPPI Method T 576 pm-07.

In another aspect, the CD wet tensile is at least 500 g/3 in.

In some aspects, the pH-modifying composition is malic acid, citric acid, hydrochloric acid, or any combination thereof.

In other aspects, the pH-modifying composition is a wetting composition.

Yet, in other aspects, the wetting composition comprises an additive, and the additive is a skin-care additive, an odor control agent, a disinfectant, a particulate, an antimicrobial agent, a preservative, a wetting agent, a cleaning agent, an emollient, a surface feel modifier, a fragrance, a fragrance solubilizer, an opacifier, a fluorescent whitening agent, an ultraviolet light absorber, or any combination thereof.

Still yet, in other aspects, the wetting pH is in a range from about 4.5 to about 5.5.

In one or more aspects, a multi-ply wet wipe including the water dispersible web has a CD wet tensile is in a range from about 75 to about 500 g/in after 24 hours in the pH-modifying composition, as measured according to IVDA Standard Test WSP 110.4 (05).

In another aspect, the CD wet tensile is in a range from about 100 to about 500 Win after 24 hours.

In some aspects, the natural cellulosic fibers are softwood fibers, hardwood fibers, or a combination thereof.

In other aspects, the glyoxalated wet strength agent is glyoxalated polyacrylamide.

Yet, in some aspects, the CMF has a length-weighted average length after fibrillation in a range from about 1.5 to about 5.0 mm.

Still yet, in other aspects, the web includes the CMF in an amount in a range from about 10 wt. % to about 50 wt. %.

In other aspects, the glyoxalated wet strength agent is present in an amount in a range from about 0.01 to about 8 pounds per ton (lb/t).

According to one or more aspects, a method of making a water dispersible wet wipe includes forming a web including CMF, a wet strength agent, and natural cellulosic fibers, the CMF being present in an amount of at least 10 wt. % based on the total weight of the web, and the remaining fibers of the web comprising natural cellulosic fibers; and disposing a pH-modifying composition having a wetting pH within the web; wherein the water dispersible wet wipe disperses upon exposure to an aqueous environment having a pH greater than the wetting pH and has a CD wet tensile of at least 200 grams/3 inch (g/3 in) as measured according to TAPPI Method T 576 pm-07.

In another aspect, the CD wet tensile is at least 500 g/3 in.

In some aspects, forming the web includes forming on a perforated belt.

In other aspects, the pH-modifying composition is malic acid, citric acid, hydrochloric acid, or any combination thereof.

Yet, in other aspects, the pH-modifying composition is a wetting composition.

Still yet, in other aspects, the wetting pH is in a range from about 3.5 to about 6.5.

In one or more aspects, the method further includes disposing another web on the web to form a multi-ply wet wipe, and the multi-ply wet wipe has a CD wet tensile in a range from about 75 to about 500 g/in after 24 hours.

In another aspect, the CD wet tensile is in a range from about 100 to about 500 Win.

In other aspects, the web has a basis weight in a range from about 25 to about 35 gsm.

In some aspects, the wet strength agent is a cationic glyoxalated wet strength agent.

Yet, in other aspects, the natural cellulosic fibers are softwood fibers, hardwood fibers, or a combination thereof.

Still yet, in some aspects, the natural cellulosic fibers are non-wood fibers.

In one or more aspects, the CMF has a length-weighted average length after fibrillation in a range from about 1.5 to about 5.0 mm.

In other aspects, the web includes the CMF in an amount in a range from about 10 wt. % to about 50 wt. %.

According to one or more aspects, a water dispersible wipe includes a web including a CMF in a range from about 10 wt. % to about 100 wt. % based on the total weight of the web, the remaining fibers of the web including natural cellulosic fibers, and the CMF having a freeness of less than 175 milliliters (mL); a glyoxalated wet strength agent disposed within the web; and a pH-modifying composition having a wetting pH disposed within the web; wherein the water dispersible wipe disperses upon exposure to an aqueous environment having a pH greater than the wetting pH and has a MD dry tensile to CD dry tensile ratio of about 1:1 to about 3:1.

In other aspects, the water dispersible wipe has a MD dry tensile to CD dry tensile ratio of about 1.5:1 to 3:1.

In some aspects, the water dispersible wipe has a MD dry tensile to CD dry tensile ratio of about 2:1 to 3:1.

Yet, in other aspects, the web includes the CMF in an amount in a range from about 10 wt. % to about 50 wt. %.

With respect to the above description then, it is to be realized that the optimum dimensional relationships for the parts of the invention, to include variations in size, materials, shape, form, function, and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, various modifications may be made of the invention without departing from the scope thereof and it is desired, therefore, that only such limitations shall be placed thereon as are imposed by the prior art and which are set forth in the appended claims.

What is claimed is:

1. A water dispersible wipe comprising:
a web comprising at least 10 weight % (wt. %) of a fibrillated regenerated cellulose microfiber (CMF) based on the total weight of the web, the remaining fibers of the web comprising natural cellulosic fibers;
a wet strength agent disposed within the web; and
a pH-modifying composition having a wetting pH disposed within the web;
wherein the water dispersible wipe comprises at least two webs and disperses upon exposure to an aqueous environment having a pH greater than the wetting pH and has a cross-direction (CD) wet tensile of at least 75 g/in after 24 hours as measured according to INDA Standard Test WSP 110.4 (05).

2. The water dispersible wipe of claim 1, wherein the pH-modifying composition is malic acid, citric acid, hydrochloric acid, sulfuric acid, or any combination thereof.

3. The water dispersible wipe of claim 1, wherein the pH-modifying composition is a wetting composition.

4. The water dispersible wipe of claim 1, wherein the wetting pH is in a range from about 3.5 to about 6.5.

5. The water dispersible wipe of claim 1, wherein the wet strength agent is a glyoxalated wet strength agent.

6. The water dispersible wipe of claim 1, wherein the natural cellulosic fibers are non-wood fibers.

7. The water dispersible wipe of claim 1, wherein the CMF has a length-weighted average length after fibrillation in a range from about 1.5 to about 5.0 millimeters (mm).

8. The water dispersible wipe of claim 1, wherein the web comprises the CMF in an amount in a range from about 10 wt. % to about 50 wt. %.

9. A water dispersible wet wipe comprising:
a web comprising CMF in a range from about 10 wt. % to about 100 wt. % based on the total weight of the web, the remaining fibers of the web comprising natural cellulosic fibers, and the CMF having a freeness of less than 175 milliliters (mL);
a glyoxalated wet strength agent disposed within the web; and
a pH-modifying composition having a wetting pH disposed within the web;
wherein the water dispersible wet wipe disperses upon exposure to an aqueous environment having a pH greater than the wetting pH and has a CD wet tensile of at least 200 grams/3 inch (g/3 in) as measured according to TAPPI Method T 576 pm-07.

10. The water dispersible wet wipe of claim 9, wherein the pH-modifying composition is a wetting composition.

11. The water dispersible wet wipe of claim 9, wherein the wetting pH is in a range from about 4.5 to about 5.5.

12. A multi-ply wet wipe comprising the water dispersible web of claim 9, wherein the CD wet tensile is in a range from about 75 to about 500 g/in after 24 hours in the pH-modifying composition, as measured according to IVDA Standard Test WSP 110.4 (05).

13. The water dispersible wet wipe of claim 9, wherein the glyoxalated wet strength agent is glyoxalated polyacrylamide.

14. The water dispersible wet wipe of claim 9, wherein the CMF has a length-weighted average length after fibrillation in a range from about 1.5 to about 5.0 mm.

15. The water dispersible wet wipe of claim 9, wherein the web comprises the CMF in an amount in a range from about 10 wt. % to about 50 wt. %.

16. The water dispersible wet wipe of claim 9, wherein the glyoxalated wet strength agent is present in an amount in a range from about 0.01 to about 8 pounds per ton (lb/T).

17. A water dispersible wipe comprising:
a web comprising CMF in a range from about 10 wt. % to about 100 wt. % based on the total weight of the web, the remaining fibers of the web comprising natural cellulosic fibers, and the CMF having a freeness of less than 175 milliliters (mL);
a glyoxalated wet strength agent disposed within the web; and
a pH-modifying composition having a wetting pH disposed within the web;
wherein the water dispersible wipe disperses upon exposure to an aqueous environment having a pH greater than the wetting pH and has a MD dry tensile to CD dry tensile ratio of about 1:1 to about 3:1.

18. The water dispersible wipe of claim 17, wherein the water dispersible wipe has a MD dry tensile to CD dry tensile ratio of about 1.5:1 to 3:1.

19. The water dispersible wipe of claim 17, wherein the water dispersible wipe has a MD dry tensile to CD dry tensile ratio of about 2:1 to 3:1.

20. The water dispersible wipe of claim 17, wherein the web comprises the CMF in an amount in a range from about 10 wt. % to about 50 wt. %.
